(19) United States
(12) Patent Application Publication
(10) Pub. No.: US 2007/0042368 A1
(43) Pub. Date: Feb. 22, 2007

(54) Detection and monitoring of lung cancer

(75) Inventors: Barbara K. Zehentner-Wilkinson, Poulsbo, WA (US); Dawn C.J. Hayes, Preston, WA (US); Raymond Houghton, Bothell, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Corixa Corporation, Seattle, WA (US)

(21) Appl. No.: 10/550,797

(22) PCT Filed: Mar. 10, 2004
(86) PCT No.: PCT/US04/07451

§ 371 Date: Jul. 26, 2006

Related U.S. Application Data
(60) Provisional application No. 60/457,261, filed on Mar. 24, 2003.
(60) Provisional application No. 60/502,995, filed on Sep. 15, 2003.

(51) Int. Cl. C12Q 1/68; C12P 19/34

(52) U.S. Cl. 435/6; 435/91.2

(57) ABSTRACT

Compositions and methods for the diagnosis of lung cancer are disclosed. Such methods are useful to detect early tumors or provide adequate stage/grade information or tumor specificity. Compositions may comprise one or more lung tumor proteins, immunogenic portions thereof, or polynucleotides that encode such portions. Such compositions may be used, for example, to improve lung cancer diagnosis and prognosis and potentially differentiate between NSCLC and SCLC.

### TECHNICAL FIELD OF THE INVENTION

The present invention relates generally to the field of cancer diagnostics. More specifically, the present invention relates to methods, compositions and kits for the detection of lung cancer in patients with different type, stage and grade of tumors that employ oligonucleotide hybridization and/or amplification to simultaneously detect two or more tissue-specific polynucleotides in a biological sample suspected of containing lung cancer cells.

### BACKGROUND OF THE INVENTION

1. Field of the Invention

Lung cancer remains a significant health problem throughout the world. The failure of conventional lung cancer treatment regimens can commonly be attributed, in part, to delayed disease diagnosis. Although significant advances have been made in the area of lung cancer diagnosis, there still remains a need for improved detection methodologies that permit early, reliable and sensitive determination of the presence of lung cancer cells.

2. Description of the Related Art

Lung cancer has the highest mortality rate of any of the cancers and is one of the most difficult to diagnose early. There are an estimated 1 million deaths annually worldwide for this disease. According to the American Cancer Society in 2002 alone there were an estimated 169,200 new cases diagnosed and ˜154,900 deaths. Typically lung cancers are classified into two major types: Non-Small Cell Lung Carcinomas (NSCLC) comprising squamous, adeno and large cell carcinomas and Small Cell Lung Carcinomas (SCLC). These groups represent ˜75% and 25% of all lung tumors respectively with adenocarcinoma and squamous cell carcinoma being the most prevalent forms of NSCLC with large cell carcinomas being ˜10%. Within the group of NSCLC, adenocarcinoma is currently the most prominent form of lung cancer in younger persons, women of all ages, lifetime nonsmokers and long-term former smokers. SCLC typically fall into two subtypes oat cell and intermediate cell. Less common tumors include carcinoid and mesotheliomas among others but these represent only a small percentage of all lung tumors. In almost all cases early diagnosis of NSCLC is elusive and most lung cancers have already metastasized by the time they are detected. Only 16.7% are localized on initial diagnosis. If tumors can be detected at a point where they are confined then the combination of chemotherapy and radiation has a possibility of success but overall the 5year prognosis is very poor with only 10-15% survival rate. The picture with SCLC is even bleaker only 6% localized at initial diagnosis and with 5 year survival rates of ˜6%.

X-ray and computer tomography of the chest and abdomen are frequently used in diagnosis of lung tumors but lack sensitivity for detecting small foci and usually detect tumors that have already metastasized. Sputum cytology as a potential screening method in high-risk individuals has only been partially effective and often does not yield tumor type. To stage the disease CAT scan, MRI or bone scans are used to evaluate the spread of disease. Treatment for lung cancer is typically surgical, radiological or chemotherapy or combinations thereof, but usually with poor outcome due to the late diagnosis of disease.

The current tests for lung cancer lack either the clinical sensitivity to detect early tumors or provide inadequate stage/grade information or lack tumor specificity due to their originating from other tumor types or being present in benign lung disorders. There is therefore a need to develop specific tests that can improve lung cancer diagnosis and prognosis and potentially differentiate between NSCLC and SCLC. The present invention achieves these and other related objectives by providing methods that are useful for the identification of tissue-specific polynucleotides, in particular tumor-specific polynucleotides, as well as antibodies and methods, compositions and kits for the detection and monitoring of cancer cells in a patient afflicted with the disease.

### SUMMARY OF THE INVENTION

The present invention provides methods for detecting the presence of lung cancer cells in a patient. Such methods comprise the steps of: (a) obtaining a biological sample from the patient; (b) contacting the biological sample with two or more oligonucleotide pairs specific for independent polynucleotide sequences which are unrelated to one another, wherein the oligonucleotide pairs hybridize, under moderately stringent conditions, to their respective polynucleotides and the complements thereof (c) amplifying the polynucleotides; and (d) detecting the amplified polynucleotides; wherein the presence of one or more of the amplified polynucleotides indicates the presence of lung cancer cells in the patient.

By some embodiments, detection of the amplified polynucleotides may be preceded by a fractionation step such as, for example, gel electrophoresis. Alternatively or additionally, detection of the amplified polynucleotides may be achieved by hybridization of a labeled oligonucleotide probe that hybridizes specifically, under moderately stringent conditions, to such polynucleotides. Oligonucleotide labeling may be achieved by incorporating a radiolabeled nucleotide or by incorporating a fluorescent label.

In certain preferred embodiments, cells of a specific tissue type may be enriched from the biological sample prior to the steps of detection. Enrichment may be achieved by a methodology selected from the group consisting of cell capture and cell depletion. Exemplary cell capture methods include immunocapture and comprise the steps of: (a) adsorbing an antibody to a tissue-specific cell surface to cells said biological sample; (b) separating the antibody adsorbed tissue-specific cells from the remainder of the biological sample. Exemplary cell depletion may be achieved by cross-linking red cells and white cells followed by a subsequent fractionation step to remove the cross-linked cells.xxx

Alternative embodiments of the present invention provide methods for determining the presence or absence of lung cancer in a patient, comprising the steps of: (a) contacting a biological sample obtained from the patient with two or more oligonucleotides that hybridize to two or more polynucleotides that encode two or more lung tumor proteins; (b) detecting in the sample a level of at least one of the polynucleotides (such as, for example, mRNA) that hybridize to the oligonucleotides; and (c) comparing the level of polynucleotides that hybridize to the oligonucleotides with a predetermined cut-off value, and therefrom determining the presence or absence of lung cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of lung cancer in a patient, comprising the steps of: (a) contacting a biological sample obtained from a patient with two or more oligonucleotides that hybridize to two or more polynucleotides that encode lung tumor proteins; (b) detecting in the sample an amount of the polynucleotides that hybridize to the oligonucleotides; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Certain embodiments of the present invention provide that the step of amplifying said first polynucleotide and said second polynucleotide is achieved by the polymerase chain reaction (PCR).

The present invention also provides kits that are suitable for performing the detection methods of the present invention. Exemplary kits comprise oligonucleotide primer pairs each one of which specifically hybridizes to a distinct polynucleotide. Within certain embodiments, kits according to the present invention may also comprise a nucleic acid polymerase and suitable buffer.

These and other aspects of the present invention will become apparent upon reference to the following detailed description and attached drawings. All references disclosed herein are hereby incorporated by reference in their entirety as if each was incorporated individually.

### BRIEF DESCRIPTION OF SEQUENCE IDENTIFIERS

SEQ ID NO: 1 is the determined cDNA sequence L762P.
SEQ ID NO: 2 is the amino acid sequence encoded by the sequence of SEQ ID NO: 1.
SEQ ID NO: 3 is the determined cDNA sequence L984P.
SEQ ID NO: 4 is the amino acid sequence encoded by the sequence of SEQ ID NO: 3.
SEQ ID NO: 5 is the determined cDNA sequence L550S.
SEQ ID NO: 6 is the amino acid sequence encoded by the sequence of SEQ ID NO: 5.
SEQ ID NO: 7 is the determined cDNA sequence L552S.
SEQ ID NO: 8 is the amino acid sequence encoded by the sequence of SEQ ID NO: 7.
SEQ ID NO:9 is the DNA sequence of L552S INT forward primer.
SEQ ID NO:10 is the DNA sequence of L552S INT reverse primer.
SEQ ID NO:11 is the DNA sequence of L552S Taqman probe.
SEQ ID NO:12 is the DNA sequence of L550S INT forward primer.
SEQ ID NO:13 is the DNA sequence of L550S INT reverse primer.
SEQ ID NO:14 is the DNA sequence of L550S Taqman probe.
SEQ ID NO:15 is the DNA sequence of L726P INT forward primer.
SEQ ID NO:16 is the DNA sequence of L726P INT reverse primer.
SEQ ID NO:17 is the DNA sequence of L726P Taqman probe.
SEQ ID NO:18 is the DNA sequence of L984P INT forward primer.
SEQ ID NO:19 is the DNA sequence of L984P INT reverse primer.
SEQ ID NO:20 is the DNA sequence of L984P Taqman probe.
SEQ ID NO:21 is the determined cDNA sequence of L763P.
SEQ ID NO:22 is the amino acid sequence encoded by the sequence of SEQ ID NO:21.
SEQ ID NO:23 is the DNA sequence of L763P INT forward primer.
SEQ ID NO:24 is the DNA sequence of L763P reverse primer.
SEQ ID NO:25 is the DNA sequence of L763P Taqman probe.
SEQ ID NO:26 is the determined cDNA sequence of L587.
SEQ ID NO:27 is the amino acid sequence encoded by the sequence of SEQ ID NO:26.
SEQ ID NO:28 is the DNA sequence of L587 INT forward primer.
SEQ ID NO:29 is the DNA sequence of L587 INT reverse primer.
SEQ ID NO:30 is the DNA sequence of L587 Taqman probe.
SEQ ID NO:31 is the determined cDNA sequence of L523.
SEQ ID NO:32 is the amino acid sequence encoded by the sequence of SEQ ID NO:31.
SEQ ID NO:33 is the DNA sequence of L523 primer.
SEQ ID NO:34 is the DNA sequence of L523 primer.

### DETAILED DESCRIPTION OF THE INVENTION

As noted above, the present invention is directed generally to methods that are suitable for the identification of tissue-specific polynucleotides as well as to methods, compositions and kits that are suitable for the diagnosis and monitoring of lung cancer, in particular such methods, compositions and kits are suitable for use in the diagnosis, differentiation and/or prognosis of NSCLC and SCLC. Such diagnostic methods will form the basis for a molecular diagnostic test for detecting lung cancer metastases in lung tissue and for the detection of anchorage independent lung cancer cells in blood as well as in mediastinal lymph nodes of distant metastases.

A variety of genes have been identified as over-expressed in lung tumors, in particular squamous or adeno forms of NSCLC or small cell carcinomas. These include, but are not limited to: L762P, L984P, L550S/L548S, L552S/L547S, L552S/L547S, L200T, L514S, L551S, L587S, L763S, L773P, L801P. L985P, L1058C, L1081C, L523S, OF1783P, B307D (WIPO International Patent Application Nos: WO 99/47674,published Sep. 23, 1999; WO 00/61612, published Oct. 19, 2000; WO 02/00174, published Jan. 3, 2002; WO 02/47534, published Jun. 20, 2002; WO 01/72295, published Oct. 4, 2001; WO 02/092001, published Nov. 21, 2002; WO 01/00828, published Jan. 1, 2001; WO 02/04514, published Jan. 17, 2002; WO 01/92525, published Dec. 6, 2002; WO 02/02623, published Jan. 10, 2002. U.S. Pat. No.: Wang et al., U.S. Pat. No. 6,482,597, issued Nov. 22, 2002; Wang et al., U.S. Pat. No. 6,518,256, issued Feb. 11, 2003; Wang et al., U.S. Pat. No. 6,426,072, issued Jul. 30, 2002; Reed et al., U.S. Pat. No. 6,210,883, issued Apr. 3, 2001; Wang et al., U.S. Pat. No. 6,504,010, issued Jan. 7, 2003; Wang et al., U.S. Pat. No. 6,509,448, issued Jan. 21, 2003. Wang et al; Oncogene; 21(49):7598-604, 2002 (collagen type XI alpha 1).).

These genes were identified and characterized using PCR and cDNA library subtractions as well as electronic subtractions with each of the tumor types individually. The cDNAs identified were then evaluated by microarray then by Real Time PCR on tissue panels to identify specific expression patterns. Table 1 highlights the specificity of these genes for either adeno or squamous forms of NSCLC or both as well as genes specific for small cell lung carcinomas. In some cases reactivity with large cell carcinomas has also been identified by Real Time PCR analysis.

TABLE 1

| | | | | | Normal |
|---|---|---|---|---|---|
| Gene | Squamous | Adeno | Small cell | Large cell | Lung |
| L762P | +++++ | + | | | − |
| L984P | | + | +++ | | − |
| L550S/L548S | | +++++ | + | | − |
| L552S/L547S | ++ | +++++ | | | − |
| L200T | + | ++ | | ++ | − |
| L514S | ++++ | ++++ | | | − |
| L551S | | ++++ | | +/− | − |
| L587S | + | + | +++ | + | − |
| L763P | +++++ | | | | − |
| L773P | +++ | +++ | | | − |
| L801P | ++++ | ++++ | | ++ | − |
| L978P | + | ++ | +++++ | +/− | − |
| L985P | | + | +++++ | | − |
| L1058C | | | ++ | | − |
| L1081C | | | ++ | | − |
| L523S | +++++ | +++++ | + | ++ | − |
| OF 1783P | | | +++++ | | − |
| B307D | ++ | ++ | | + | − |

Identification of Tissue-specific Polynucleotides

Certain embodiments of the present invention provide methods, compositions and kits for the detection of lung cancer cells within a biological sample from patients with different type, stage and grade of tumors. These methods comprise the step of detecting one or more tissue-specific polynucleotide(s) from a patient's biological sample the over-expression of which polynucleotides indicates the presence of lung cancer cells within the patient's biological sample. Accordingly, the present invention also provides methods that are suitable for the identification of tissue-specific polynucleotides. As used herein, the phrases “tissue-specific polynucleotides” or “tumor-specific polynucleotides” are meant to include all polynucleotides that are at least two-fold over-expressed as compared to one or more control tissues. As discussed in further detail herein below, over-expression of a given polynucleotide may be assessed, for example, by microarray and/or quantitative real-time polymerase chain reaction (Real-time PCR™) methodologies.

Exemplary methods for detecting tissue-specific polynucleotides may comprise the steps of: (a) performing a genetic subtraction to identify a pool of polynucleotides from a tissue of interest; (b) performing a DNA microarray analysis to identify a first subset of said pool of polynucleotides of interest wherein each member polynucleotide of said first subset is at least two-fold over-expressed in said tissue of interest as compared to a control tissue; and (c) performing a quantitative polymerase chain reaction analysis on polynucleotides within said first subset to identify a second subset of polynucleotides that are at least two-fold over-expressed as compared to said control tissue.

Polynucleotides Generally

As used herein, the term “polynucleotide” refers generally to either DNA or RNA molecules. Polynucleotides may be naturally occurring as normally found in a biological sample such as blood, serum, lymph node, bone marrow, sputum, urine and tumor biopsy samples. Alternatively, polynucleotides may be derived synthetically by, for example, a nucleic acid polymerization reaction. As will be recognized by the skilled artisan, polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e. an endogenous sequence that encodes a tumor protein, such as a lung tumor protein, or a portion thereof) or may comprise a variant, or a biological or antigenic functional equivalent of such a sequence. Polynucleotide variants may contain one or more substitutions, additions, deletions and/or insertions, as further described below. The term “variants” also encompasses homologous genes of xenogenic origin.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (ie., the window size) and multiplying the results by 100 to yield the percentage of sequence identity.

Therefore, the present invention encompasses polynucleotide and polypeptide sequences having substantial identity to the sequences disclosed herein, for example those comprising at least 50% sequence identity, preferably at least 55%, 60%, 65%, 70%, 75%, 80%, 85%, 90%, 95%, 96%, 97%, 98%, or 99% or higher, sequence identity compared to a polynucleotide or polypeptide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

In other embodiments, the present invention is directed to polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Microarray Analyses

Polynucleotides that are suitable for detection according to the methods of the present invention may be identified, as described in more detail below, by screening a microarray of cDNAs for tissue and/or tumor-associated expression (e.g., expression that is at least two-fold greater in a tumor than in normal tissue, as determined using a representative assay provided herein). Such screens may be performed, for example, using a Synteni microarray (Palo Alto, Calif.) according to the manufacturer's instructions (and essentially as described by Schena et al., Proc. Natl. Acad. Sci. USA 93:10614-10619 (1996) and Heller et al., Proc. Natl. Acad. Sci. USA 94:2150-2155 (1997)).

Microarray is an effective method for evaluating large numbers of genes but due to its limited sensitivity it may not accurately determine the absolute tissue distribution of low abundance genes or may underestimate the degree of overexpression of more abundant genes due to signal saturation. For those genes showing overexpression by microarray expression profiling, further analysis was performed using quantitative RT-PCR based on Taqman™ probe detection, which comprises a greater dynamic range of sensitivity. Several different panels of normal and tumor tissues, distant metastases and cell lines were used for this purpose.

Quantitative Real-time Polymerase Chain Reaction

Suitable polynucleotides according to the present invention may be further characterized or, alternatively, originally identified by employing a quantitative PCR methodology such as, for example, the Real-time PCR methodology. By this methodology, tissue and/or tumor samples, such as, e.g., metastatic tumor samples, may be tested along side the corresponding normal tissue sample and/or a panel of unrelated normal tissue samples.

Real-time PCR (see Gibson et al., Genome Research 6:995-1001, 1996; Heid et al., Genome Research 6:986-994, 1996) is a technique that evaluates the level of PCR product accumulation during amplification. This technique permits quantitative evaluation of mRNA levels in multiple samples. Briefly, mRNA is extracted from tumor and normal tissue and cDNA is prepared using standard techniques.

Real-time PCR may, for example, be performed either on the ABI 7700 Prism or on a GeneAmp® 5700 sequence detection system (Applied Biosystems, Foster City, Calif.). The 7700 system uses a forward and a reverse primer in combination with a specific probe with a 5′ fluorescent reporter dye at one end and a 3′ quencher dye at the other end (Taqman™). When the Real-time PCR is performed using Taq DNA polymerase with 5′ -3′ nuclease activity, the probe is cleaved and begins to fluoresce allowing the reaction to be monitored by the increase in fluorescence (Real-time). The 5700 system uses SYBR® green, a fluorescent dye, that only binds to double stranded DNA, and the same forward and reverse primers as the 7700 instrument. Matching primers and fluorescent probes may be designed according to the primer express program (Applied Biosystems, Foster City, Calif.). Optimal concentrations of primers and probes are initially determined by those of ordinary skill in the art. Control (e.g., β-actin) primers and probes may be obtained commercially from, for example, Perkin Elmer/Applied Biosystems (Foster City, Calif.).

To quantitate the amount of specific RNA in a sample, a standard curve is generated using a plasmid containing the gene of interest. Standard curves are generated using the Ct values determined in the real-time PCR, which are related to the initial cDNA concentration used in the assay. Standard dilutions ranging from 10-10⁶ copies of the gene of interest are generally sufficient. In addition, a standard curve is generated for the control sequence. This permits standardization of initial RNA content of a tissue sample to the amount of control for comparison purposes.

In accordance with the above, and as described further below, the present invention provides the illustrative lung tissue- and/or tumor-specific polynucleotides L552S, L550S, L762P, L984P, L763P and L587 having sequences set forth in SEQ ID NOs: 1, 3, 5, 7, 21 and 26, illustrative polypeptides encoded thereby having amino acid sequences set forth in SEQ ID NO: 2, 4, 6, 8, 22 and 27 that may be suitably employed in the detection of cancer, more specifically , lung cancer.

Methodologies for the Detection of Cancer

In general, a cancer cell may be detected in a patient based on the presence of one or more polynucleotides within cells of a biological sample (for example, blood, lymph nodes, bone marrow, sera, sputum, urine and/or tumor biopsies) obtained from the patient. In other words, such polynucleotides may be used as markers to indicate the presence or absence of a cancer such as, e.g., lung cancer.

As discussed in further detail herein, the present invention achieves these and other related objectives by providing a methodology for the simultaneous detection of more than one polynucleotide, the presence of which is diagnostic of the presence of lung cancer cells in a biological sample. Each of the various cancer detection methodologies disclosed herein have in common a step of hybridizing one or more oligonucleotide primers and/or probes, the hybridization of which is demonstrative of the presence of a tumor- and/or tissue-specific polynucleotide. Depending on the precise application contemplated, it may be preferred to employ one or more intron-spanning oligonucleotides that are inoperative against polynucleotide of genomic DNA and, thus, these oligonucleotides are effective in substantially reducing and/or eliminating the detection of genomic DNA in the biological sample.

Further disclosed herein are methods for enhancing the sensitivity of these detection methodologies by subjecting the biological samples to be tested to one or more cell capture and/or cell depletion methodologies.

By certain embodiments of the present invention, the presence of lung cancer cell in a patient may be determined by employing the following steps: (a) contacting a biological sample obtained from the patient with two or more oligonucleotides that hybridize to two or more polynucleotides that encode two or more lung tumor proteins as described herein; (b) detecting in the sample a level of at least one of the polynucleotides (such as, for example, mRNA) that hybridize to the oligonucleotides; and (c) comparing the level of polynucleotides that hybridize to the oligonucleotides with a predetermined cut-off value, and therefrom determining the presence or absence of lung cancer in the patient.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a lung tumor protein that is at least 10 nucleotides, and preferably at least 20 nucleotides, in length. Preferably, oligonucleotide primers hybridize to a polynucleotide encoding a polypeptide described herein under moderately stringent conditions, as defined above. Oligonucleotide primers which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence recited in SEQ ID NO: 1, 3, 5 or 7. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, N.Y., 1989).

The present invention also provides amplification-based methods for detecting the presence of lung cancer cells in a patient. Exemplary methods comprise the steps of (a) obtaining a biological sample from the patient; (b) contacting the biological sample with two or more oligonucleotide pairs specific for independent polynucleotide sequences which are unrelated to one another, wherein the oligonucleotide pairs hybridize, under moderately stringent conditions, to their respective polynucleotides and the complements thereof (c) amplifying the polynucleotides; and (d) detecting the amplified polynucleotides; wherein the presence of one or more of the amplified polynucleotides indicates the presence of lung cancer cells in the patient.

Methods according to the present invention are suitable for identifying polynucleotides obtained from a wide variety of biological sample such as, for example, blood, serum, lymph node, bone marrow, sputum, urine and tumor biopsy sample, among others.

Certain exemplary embodiments of the present invention provide methods wherein the polynucleotides to be detected are selected from the group consisting of L762, L984, L550, L552, L763 and L587. Alternatively and/or additionally, polynucleotides to be detected may be selected from the group consisting of those depicted in SEQ ID NOs: 1, 3, 5, 7, 21 and 26.

Suitable exemplary oligonucleotide probes and/or primers that may be used according to the methods of the present invention are disclosed herein. In certain preferred embodiments that eliminate the background detection of genomic DNA, the oligonucleotides may be intron spanning oligonucleotides.

Depending on the precise application contemplated, the artisan may prefer to detect the tissue- and/or tumor-specific polynucleotides by detecting a radiolabel and detecting a fluorophore. More specifically, the oligonucleotide probe and/or primer may comprises a detectable moiety such as, for example, a radiolabel and/or a fluorophore.

Alternatively or additionally, methods of the present invention may also comprise a step of fractionation prior to detection of the tissue- and/or tumor-specific polynucleotides such as, for example, by gel electrophoresis.

In other embodiments, methods described herein may be used as to monitor the progression of cancer. By these embodiments, assays as provided for the diagnosis of lung cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple lung tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

Cell Enrichment

In other aspects of the present invention, cell capture technologies may be used prior to polynucleotide detection to improve the sensitivity of the various detection methodologies disclosed herein.

Exemplary cell enrichment methodologies employ immunomagnetic beads that are coated with specific monoclonal antibodies to surface cell markers, or tetrameric antibody complexes, may be used to first enrich or positively select cancer cells in a sample. Various commercially available kits may be used, including Dynabeads® Epithelial Enrich (Dynal Biotech, Oslo, Norway), StemSep™ (StemCell Technologies, Inc., Vancouver, BC), and RosetteSep (StemCell Technologies). The skilled artisan will recognize that other readily available methodologies and kits may also be suitably employed to enrich or positively select desired cell populations.

Dynabeads® Epithelial Enrich contains magnetic beads coated with mAbs specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses.

RosetteSep can be used to enrich cells directly from a blood sample and consists of a cocktail of tetrameric antibodies that target a variety of unwanted cells and crosslinks them to glycophorin A on red blood cells (RBC) present in the sample, forming rosettes. When centrifuged over Ficoll, targeted cells pellet along with the free RBC.

The combination of antibodies in the depletion cocktail determines which cells will be removed and consequently which cells will be recovered. Antibodies that are available include, but are not limited to: CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE, and TCRαβ. Additionally, it is contemplated in the present invention that mAbs specific for lung tumor antigens, can be developed and used in a similar manner. For example, mAbs that bind to tumor-specific cell surface antigens may be conjugated to magnetic beads, or formulated in a tetrameric antibody complex, and used to enrich or positively select metastatic lung tumor cells from a sample. Such a system can be used to evaluate blood samples from different forms of lung cancers, in particular adneo and squamous forms of NSCLC and small cell carcinomas for the presence of circulating tumor cells using the inventive multiplex PCR assay as described herein.

Once a sample is enriched or positively selected, cells may be further analyzed. For example, the cells may be lysed and RNA isolated. RNA may then be subjected to RT-PCR analysis using lung tumor-specific multiplex primers in a Real-time PCR assay as described herein.

In another aspect of the present invention, cell capture technologies may be used in conjunction with Real-Time PCR to provide a more sensitive tool for detection of metastatic cells expressing lung tumor antigens.

Yet another method that may be employed is an anti-ganglioside G_{M1}/G_{M1} cell capture antibody system. Gangliosides are cell membrane bound glycosphingolipids, several species of which have been shown to be over-expressed on the cell surface of most cancers of neuroectodermal and epithelial origin, in particular lung cancer. Cell surface expression of G_{M2} is seen in several types of lung cancer, particularly in SCLC which make it an attractive target for a monoclonal antibody based lung cancer immunotherapy and also for use as a capture method in conjunction with G_{M1}.

Probes and Primers

As noted above and as described in further detail herein, certain methods, compositions and kits according to the present invention utilize two or more oligonucleotide primer pairs for the detection of lung cancer. The ability of such nucleic acid probes to specifically hybridize to a sequence of interest will enable them to be of use in detecting the presence of complementary sequences in a biological sample.

Alternatively, in other embodiments, the probes and/or primers of the present invention may be employed for detection via nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence of a polynucleotide to be detected will find particular utility. Longer contiguous identical or complementary sequences, e.g., those of about. 20, 30, 40, 50, 100, 200, 500, 1000 (including all intermediate lengths) and even up to full length sequences will also be of use in certain embodiments.

Oligonucleotide primers having sequence regions consisting of contiguous nucleotide stretches of 10-14, 15-20, 30, 50, or even of 100-200 nucleotides or so (including intermediate lengths as well), identical or complementary to a polynucleotide to be detected, are particularly contemplated as primers for use in amplification reactions such as, e.g., the polymerase chain reaction (PCR™). This would allow a polynucleotide to be analyzed, both in diverse biological samples such as, for example, blood, lymph nodes and bone marrow.

The use of a primer of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design primers having gene-complementary stretches of 15 to 25 contiguous nucleotides, or even longer where desired.

Primers may be selected from any portion of the polynucleotide to be detected. All that is required is to review the sequence, such as those exemplary polynucleotides set forth herein or to any continuous portion of the sequence, from about 15-25 nucleotides in length up to and including the full length sequence, that one wishes to utilize as a primer. The choice of primer sequences may be governed by various factors. For example, one may wish to employ primers from towards the termini of the total sequence. The exemplary primers disclosed herein may optionally be used for their ability to selectively form duplex molecules with complementary stretches of the entire polynucleotide of interest such as those set forth SEQ ID NOs: 1, 3, 5, 7, 21 and 26.

The present invention further provides the nucleotide sequence of various exemplary oligonucleotide primers and probes, that may be used, as described in further detail herein, according to the methods of the present invention for the detection of cancer.

Oligonucleotide primers according to the present invention may be readily prepared routinely by methods commonly available to the skilled artisan including, for example, directly synthesizing the primers by chemical means, as is commonly practiced using an automated oligonucleotide synthesizer. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Polynucleotide Amplification Techniques

Each of the specific embodiments outlined herein for the detection of lung cancer has in common the detection of a tissue- and/or tumor-specific polynucleotide via the hybridization of one or more oligonucleotide primers and/or probes. Depending on such factors as the relative number of cancer cells present in the biological sample and/or the level of polynucleotide expression within each lung cancer cell, it may be preferred to perform an amplification step prior to performing the steps of detection. For example, at least two oligonucleotide primers may be employed in a polymerase chain reaction (PCR) based assay to amplify a portion of a lung tumor cDNA derived from a biological sample, wherein at least one of the oligonucleotide primers is specific for (i.e., hybridizes to) a polynucleotide encoding the lung tumor protein. The amplified cDNA may optionally be subjected to a fractionation step such as, for example, gel electrophoresis.

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

One preferred methodology for polynucleotide amplification employs RT-PCR, in which PCR is applied in conjunction with reverse transcription. Typically, RNA is extracted from a biological sample, such as blood, serum, lymph node, bone marrow, sputum, urine and tumor biopsy samples, and is reverse transcribed to produce cDNA molecules. PCR amplification using at least one specific primer generates a cDNA molecule, which may be separated and visualized using, for example, gel electrophoresis. Amplification may be performed on biological samples taken from a patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

Any of a variety of commercially available kits may be used to perform the amplification step. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WIPO International Patent Application No.: WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1: 111-19, 1991) and walking PCR (Parker et al., Nucl. Acids. Res. 19:3055-60, 1991). Other methods employing amplification may also be employed to obtain a full length cDNA sequence.

Another method for amplification is the ligase chain reaction (referred to as LCR), disclosed in Eur. Pat. Appl. Publ. No. 320,308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, describes an alternative method of amplification similar to LCR for binding probe pairs to a target sequence. Qbeta Replicase, described in PCT Intl. Pat. Appl. Publ. No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]triphosphates in one strand of a restriction site (Walker et al., 1992), may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having a 3′ and 5′ sequences of non-target DNA and an internal or “Middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods described in Great Britain Pat. Appl. No. 2 202 328, and in PCT Intl. Pat. Appl. Publ. No. PCT/US89/01025, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H (RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA “dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, disclose a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “RACE” (Frohman, 1990), and “one-sided PCR” (Ohara, 1989) which are well-known to those of skill in the art.

Compositions and Kits for the Detection of Cancer

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a lung tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

The present invention also provides kits that are suitable for performing the detection methods of the present invention. Exemplary kits comprise oligonucleotide primer pairs each one of which specifically hybridizes to a distinct polynucleotide. Within certain embodiments, kits according to the present invention may also comprise a nucleic acid polymerase and suitable buffer. Exemplary oligonucleotide primers suitable for kits of the present invention are disclosed herein. Exemplary polynucleotides suitable for kits of the present invention are disclosed herein.

Alternatively, a kit may be designed to detect the level of mRNA encoding a lung tumor protein in a biological sample. Such kits generally comprise at least one oligonucleotide probe or primer, as described above, that hybridizes to a polynucleotide encoding a lung tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a lung tumor protein.

In other related aspects, the present invention further provides compositions useful in the methods disclosed herein. Exemplary compositions comprise two or more oligonucleotide primer pairs each one of which specifically hybridizes to a distinct polynucleotide. Exemplary oligonucleotide primers suitable for compositions of the present invention are disclosed herein. Exemplary polynucleotides suitable for compositions of the present invention are disclosed herein.

The following Example is offered by way of illustration and not by way of limitation.

### EXAMPLES

### Example 1

### Multiplex Detection of Lung Tumors

A Multiplex Real-time PCR assay was established in order to simultaneously detect the expression of four lung cancer-specific genes: L762 (SEQ ID NO:1), L984 (SEQ ID NO:3), L550 (SEQ ID NO:5) and L552 (SEQ ID NO:7). In contrast to detection approaches relying on expression analysis of single lung cancer-specific genes, this Multiplex assay was able to detect all lung tumor samples tested and analyze their combined mRNA expression profile in adenocarcinoma, squamous, small cell and large cell lung tumors. L552S and L550S complement each other in detecting predominantly adenocarcinomas, L762S detects squamous cell carcinomas and L984P detects small cell carcinomas (see Table 1).

The primers and probes were designed to be intron spanning (exon specific) to eliminate any reactivity with genomic DNA making them suitable for use in blood samples without having to DNAse treat mRNA samples. They were also designed to produce amlicons of different sizes to allow gel differentiation of end products if necessary.

The assay was carried out as follows: L552S (SEQ ID NO: 7), L550 (SEQ ID NO: 5), L762 (SEQ ID NO: 1), L984 (SEQ ID NO: 3) and specific primers, and specific Taqman probes, were used to analyze their combined mRNA expression profile in lung tumors. The primers and probes are shown below:

L552S:
Forward Primer (SEQ ID NO:9):
5′ GACGGCATGAGCGACACACA.
Reverse Primer (SEQ ID NO:10):
5′ CCATGTCGCGCACTGGGATC.
Probe (SEQ ID NO:11) (FAM-5′ - 3′-TAMRA):
CTGAAAGTCGGGATCCTACACCTGGGCA.
L550P:
Forward Primer (SEQ ID NO:12):
5′ GGCCACCGTCTGGATTCTTC.
Reverse Primer (SEQ ID NO:13):
5′ GAAGAATCCAGACGGTGGCC.
Probe (SEQ ID NO:14) (FAM-5′ - 3′-TAMRA):
CCGCCCCAAG ATCAAATCCA CAAACC.
L762S:
Forward Primer (SEQ ID NO:15):
5′ ATGGCAGAGGCTGACAGACTC.
Reverse Primer (SEQ ID NO:16):
5′ TTCAACCACCTCAAATCCTTTCTTA.
Probe (SEQ ID NO:17) (FAM-5′ - 3′-TAMRA):
TCGACAGCAAAGGAGAGATCAGAGCCC.
L984P:
Forward Primer (SEQ ID NO:18):
5′ TTACGACCCGCTCAGCCC.
Reverse Primer (SEQ ID NO:19):
5′ CTCCCAACGCCACTGACAA.
Probe (SEQ ID NO:20) (FAM-5′ - 3′-TAMRA):
CCAGGCCGAGCCCCTCAGAACC.

The assay conditions were:

Taqman protocol (7700 Perkin Elmer):

In 25 μl final volume: 1× Buffer A, 5 mM MgCl, 0.2 mM dCTP, 0.2 mM dATP, 0.4 mM dUTP, 0.2 mM dGTP, 0.01 U/μl AmpErase UNG, 0.0375 U/μl TaqGold, 8% (v/v) Glycerol, 0.05% (v/v) (Sigma), Gelatin, 0.05% (v/v) (Sigma), Tween 20 0.1% v/v (Sigma), 300 mM of each forward and reverse primer for L762P, 50 mM of each forward and reverse primer from (L552S, L984P, L550S, L984P) 2 pmol of each gene specific Taqman probe (L552S, L550S, L984P) and template cDNA. The PCR reaction was carried out at one cycle at 95° C. for 10 minutes, followed by 50 cycles at 95° C. for 15 seconds, 60° C. for 1 minute, and 68° C. for 1 minute (ABI Prism 7900HO Sequence Detection System, Foster City, Calif.).

Since each primer set in the multiplex assay results in a band of unique length, expression signals of the four genes of interest was measured individually by agarose gel analysis. The combined expression signal of all four genes can also be measured in real-time on an ABI 7700 Prism sequence detection system (Applied Biosystems, Foster City, Calif.). Although specific primers have been described herein, different primer sequences, different primer or probe labeling and different detection systems could be used to perform this multiplex assay. For example, a second fluorogenic reporter dye could be incorporated for parallel detection of a reference gene by real-time PCR. Or, for example a SYBR Green detection system could be used instead of the Taqman probe approach. Table 2 shows the reactivity of the multiplex PCR with different lung tumor types and normal lung tissue.

TABLE 2
Expression of Lung Cancer Multiplex Genes (L762P, L552S,
L550S, L984P) in Lung Tumor and Normal Lung

| Lung Tumor Type | Positive Samples/Samples Tested |
|---|---|
| Adenocarcinoma | 21/24 |
| Squamous | 17/18 |
| Large Cell | 5*/5 |
| Small Cell | 5/6 |
| Normal Lung Tissue | 0/12 |
| Total Tumors | 48/53 |
| % Positive Tumors | 90.57% |

Cut-off Value = Mean normal lung + 3 SD = 0.901
*One sample at cut-off

### Example 2

### Multiplex Detection of Lung Tumors

Six additional Multiplex Real-time PCR assays were established in order to simultaneously detect the expression of various combinations of recognized lung antigens: L762 (SEQ ID NO:1), L984 (SEQ ID NO:3), L550 (SEQ ID NO:5), L552 (SEQ ID NO:7), L763 (SEQ ID NO: 21) and L587 (SEQ ID NO:26). The six groups consisted of:
Group 1: L762, L552, L550 and L984
Group 2: L763, L552, L550 and L984
Group 3: L763, L552, L587 and L984
Group 4: L763, L550, L587 and L984
Group 5: L763, L550 and L587
Group 6: L762, L984, L550 and L587

The assays were carried out described above in Example 1 to analyze the combined mRNA expression profile in lung tumors. The primers and probes for L552S, L550P, L762S, L984P are as described in Example 1. primers and probes for L763 and L587 are described below:

L763S:
Forward Primer (SEQ ID NO:23):
5′ ATTCCAGGCGACATCCTCACT.
Reverse Primer (SEQ ID NO:24):
5′ GTTTATCCCTGAGTCCTGTTTCCA.
Probe (SEQ ID NO:25) (FAM-5′ - 3′-TAMRA):
TGTGCACCATTGGCTTCTAGGCACTCC.
L587:
Forward Primer (SEQ ID NO:28):
5′ CCCAGAGCTGTGTTAAGGGATC.
Reverse Primer (SEQ ID NO:29):
5′ GTTAAGCGGGATTTCATGTACGA.
Probe (SEQ ID NO:30) (FAM-5′ - 3′-TAMRA):
AGAACCTGAACCCGTAAAGAAGCCTCCC.

The lung antigens that make up the six multiplex assays are able to detect all lung tumor samples tested and were analyzed for their combined mRNA expression profile in adenocarcinoma, squamous, small cell and large cell lung tumors. The results of these assays is presented in Table 3.

TABLE 3
Expression of Lung Cancer Multiplex Genes in Lung Tumor and Normal Lung

| Lung Tumor | Positive Samples/Samples Tested | | | | | |
|---|---|---|---|---|---|---|
| Type | Group 1 | Group 2 | Group 3 | Group 4 | Group 5 | Group 6 |
| Adenocarcinoma | 21/24 | 21/24 | 20/24 | 22/24 | 22/24 | 22/24 |
| Squamous | 17/18 | 17/18 | 18/18 | 18/18 | 18/18 | 18/18 |
| Large Cell | 5/5 | 3/5 | 4/5 | 3/5 | 3/5 | 4/5 |
| Small Cell | 1/2 | 1/2 | 1/2 | 2/2 | 1/2 | 2/2 |
| Other | 2/2 | 2/2 | 2/2 | 2/2 | 2/2 | 2/2 |
| Normal Lung | 0/12 | 0/12 | 0/12 | 0/13 | 0/13 | 0/13 |
| Tissue | | | | | | |
| Total Tumors | 46/51 | 44/51 | 45/51 | 47/51 | 46/51 | 48/51 |
| % Positive | 90.20% | 86.27% | 88.24% | 92.16% | 90.20% | 94.12% |
| Tumors | CO = 0.9 | CO = 4.7 | CO = 1.08 | CO = 1.88 | CO = 2.2 | CO = 5.5 |

Cut-off Value (CO) = Mean normal lung + 3 SD

Multiplex assays using groups 1, 4 and 6 were next used to detect circulating tumor cells in peripheral blood samples from 17 lung cancer patients undergoing various types of treatments. In addition, a single gene assay using lung antigen L523 (SEQ ID NO:31) was carried out in parallel using the primers as described in SEQ ID NOs:33 and 34. Six normal donors were included as controls. The assays were carried out as described above in Example 1. The cut off value for detection in the assay being the mean of the normal lung samples +3 standard deviations.

Group 1 antigens were detected in 5/17 samples tested. Group 4 antigens were detected in 4/17 samples and Group 6 antigens were detected in 8/17 samples. L523 was detected as a single gene in 7/17 samples tested. The combination of antigens in Group 6 was the most sensitive for lung tumor detection in tissue and blood of the groups tested.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1.-4. (canceled)

5. A method for detecting the presence of lung cancer cells in a biological sample comprising the steps of:
(a) detecting the level of mRNA expression in the biological sample of two or more cancer-associated markers selected from the group consisting of L762P, L550S, L587S, L984P, L552S, and L763P; and
(b) comparing the level of mRNA expression detected in the biological sample for each marker to a predetermined cut-off value for each marker;
wherein a detected level of expression above the predetermined cut-off value for one or more markers is indicative of the presence of lung cancer cells in the biological sample.

6. A method for detecting the presence of lung cancer cells in a biological sample comprising the steps of:
(a) detecting the level of mRNA expression in the biological sample of two or more cancer-associated markers selected from the group consisting of L762P, L550S, L587S, and L984P; and
(b) comparing the level of mRNA expression detected in the biological sample for each marker to a predetermined cut-off value for each marker;
wherein a detected level of expression above the predetermined cut-off value for one or more markers is indicative of the presence of lung cancer cells in the biological sample.

7. The method of claim 6, wherein step (a) comprises detecting the level of mRNA expression using a nucleic acid hybridization technique.

8. The method of claim 6, wherein step (a) comprises detecting the level of mRNA expression using a nucleic acid amplification method.

9. The method of claim 8, wherein step (a) comprises detecting the level of mRNA expression using a nucleic acid amplification method selected from the group consisting of transcription-based amplification, polymerase chain reaction amplification (PCR), ligase chain reaction amplification (LCR), strand displacement amplification (SDA), and nucleic acid sequence based amplification (NASBA).

10. The method of claim 6, wherein the L762P cancer-associated marker comprises a nucleic acid sequence set forth in SEQ ID NO: 1 or a nucleic acid sequence encoding an amino acid sequence set forth in SEQ ID NO: 2.

11. The method of claim 6, wherein the L550S cancer-associated marker comprises a nucleic acid sequence set forth in SEQ ID NO: 5 or a nucleic acid sequence encoding an amino acid sequence set forth in SEQ ID NO: 6.

12. The method of claim 6, wherein the L587S cancer-associated marker comprises a nucleic acid sequence set forth in SEQ ID NO: 26 or a nucleic acid sequence encoding an amino acid sequence set forth in SEQ ID NO: 27.

13. The method of claim 6, wherein the L984P cancer-associated marker comprises a nucleic acid sequence set forth in SEQ ID NO: 3 or a nucleic acid sequence encoding an amino acid sequence set forth in SEQ ID NO: 4.

14. The method of claim 6, wherein the cancer is a small cell lung cancer or a non-small cell lung cancer.

15. The method of claim 6, wherein the biological sample is a sample suspected of containing cancer-associated markers or cancer cells expressing such markers.

16. The method of claim 15, wherein the biological sample is selected from the group consisting of a biopsy sample, lavage sample, sputum sample, serum sample, peripheral blood sample, lymph node sample, bone marrow sample, urine sample, and pleural effusion sample.

17. A composition for detecting cancer cells in a biological sample comprising two or more of:
a) a first oligonucleotide that specifically hybridizes to L762P;
b) a second oligonucleotide that specifically hybridizes to L550S;
c) a third oligonucleotide that specifically hybridizes to L587S; and
d) a fourth oligonucleotide that specifically hybridizes to L984P.

18. The composition of claim 17, wherein the first oligonucleotide specifically hybridizes to an L762P nucleic acid sequence set forth in SEQ ID NO: 1 or a nucleic acid sequence encoding an amino acid sequence set forth in SEQ ID NO: 2, the second oligonucleotide specifically hybridizes to an L550S nucleic acid sequence set forth in SEQ ID NO:5 or a nucleic acid sequence encoding an amino acid sequence set forth in SEQ ID NO: 6, the third oligonucleotide specifically hybridizes to an L587S nucleic acid sequence set forth in SEQ ID NO: 26 or a nucleic acid sequence encoding an amino acid sequence set forth in SEQ ID NO: 27, and the fourth oligonucleotide specifically hybridizes to an L984P nucleic acid sequence set forth in SEQ ID NO: 3 or a nucleic acid sequence encoding an amino acid sequence set forth in SEQ ID NO: 4.

19. A diagnostic kit for detecting cancer cells in a biological sample comprising two or more of:
a) a first oligonucleotide that specifically hybridizes to L762P;
b) a second oligonucleotide that specifically hybridizes to L550S;
c) a third oligonucleotide that specifically hybridizes to L587S; and
d) a fourth oligonucleotide that specifically hybridizes to L984P.

20. The kit of claim 19, wherein the first oligonucleotide specifically hybridizes to an L762P nucleic acid sequence set forth in SEQ ID NO: 1 or a nucleic acid sequence encoding an amino acid sequence set forth in SEQ ID NO: 2, the second oligonucleotide specifically hybridizes to an L550S nucleic acid sequence set forth in SEQ ID NO:5 or a nucleic acid sequence encoding an amino acid sequence set forth in SEQ ID NO: 6, the third oligonucleotide specifically hybridizes to an L587S nucleic acid sequence set forth in SEQ ID NO: 26 or a nucleic acid sequence encoding an amino acid sequence set forth in SEQ ID NO: 27, and the fourth oligonucleotide specifically hybridizes to an L984P nucleic acid sequence set forth in SEQ ID NO: 3 or a nucleic acid sequence encoding an amino acid sequence set forth in SEQ ID NO: 4.

21. A composition for detecting cancer cells in a biological sample comprising two or more of:
a) a first primer pair that specifically hybridizes to L762P;
b) a second primer pair that specifically hybridizes to L550S;
c) a third primer pair that specifically hybridizes to L587S; and
d) a fourth primer pair that specifically hybridizes to L984P.

22. The composition of claim 21, wherein the first, second, third and fourth primer pairs are effective in a nucleic acid amplification method for amplifying all or a portion of an L762P nucleic acid sequence set forth in SEQ ID NO: 1 or a nucleic acid sequence encoding an amino acid sequence set forth in SEQ ID NO: 2, an L550S nucleic acid sequence set forth in SEQ ID NO:5 or a nucleic acid sequence encoding an amino acid sequence set forth in SEQ ID NO: 6, an L587S nucleic acid sequence set forth in SEQ ID NO: 26 or a nucleic acid sequence encoding an amino acid sequence set forth in SEQ ID NO: 27, and an L984P nucleic acid sequence set forth in SEQ ID NO: 3 or a nucleic acid sequence encoding an amino acid sequence set forth in SEQ ID NO: 4, respectively.

23. A diagnostic kit for detecting cancer cells in a biological sample comprising two or more of:
a) a first primer pair that specifically hybridizes to L762P;
b) a second primer pair that specifically hybridizes to L550S;
c) a third primer pair that specifically hybridizes to L587S; and
d) a fourth primer pair that specifically hybridizes to L984P.

24. The kit of claim 23, wherein the first, second, third and fourth primer pairs are effective in a nucleic acid amplification method for amplifying all or a portion of an L762P nucleic acid sequence set forth in SEQ ID NO: 1 or a nucleic acid sequence encoding an amino acid sequence set forth in SEQ ID NO: 2, an L550S nucleic acid sequence set forth in SEQ ID NO:5 or a nucleic acid sequence encoding an amino acid sequence set forth in SEQ ID NO: 6, an L587S nucleic acid sequence set forth in SEQ ID NO: 26 or a nucleic acid sequence encoding an amino acid sequence set forth in SEQ ID NO: 27, and an L984P nucleic acid sequence set forth in SEQ ID NO: 3 or a nucleic acid sequence encoding an amino acid sequence set forth in SEQ ID NO: 4, respectively.

25. A diagnostic kit for detecting cancer cells in a biological sample comprising two or more of:
a) a first antibody specific for an L762P protein;
b) a second antibody specific for an L550S protein;
c) a third antibody specific for an L587S protein; and
d) a fourth antibody specific for an L984P protein.

26. The kit of claim 25, wherein the L762P protein comprises an amino acid sequence set forth in SEQ ID NO: 2, the L550S protein comprises an amino acid sequence set forth in SEQ ID NO: 6, the L587S protein comprises an amino acid sequence set forth in SEQ ID NO: 27, and the L984P protein comprises an amino acid sequence set forth in SEQ ID NO: 4.


SEQUENCE LISTING (34 sequences)

SEQ ID NO: 1 - DNA, 3951 bp, Homo sapiens
tctgcatccatattgaaaacctgacacaatgtatgcagcaggctcagtgtgagtgaactggaggcttctctacaacatgacccaaaggagcattgcaggtcctatttgcaacctgaagtttgtgactctcctggttgccttaagttcagaactcccattcctgggagctggagtacagcttcaagacaatgggtataatggattgctcattgcaattaatcctcaggtacctgagaatcagaacctcatctcaaacattaaggaaatgataactgaagcttcattttacctatttaatgctaccaagagaagagtatttttcagaaatataaagattttaatacctgccacatggaaagctaataataacagcaaaataaaacaagaatcatatgaaaaggcaaatgtcatagtgactgactggtatggggcacatggagatgatccatacaccctacaatacagagggtgtggaaaagagggaaaatacattcatttcacacctaatttcctactgaatgataacttaacagctggctacggatcacgaggccgagtgtttgtccatgaatgggcccacctccgttggggtgtgttcgatgagtataacaatgacaaacctttctacataaatgggcaaaatcaaattaaagtgacaaggtgttcatctgacatcacaggcatttttgtgtgtgaaaaaggtccttgcccccaagaaaactgtattattagtaagctttttaaagaaggatgcacctttatctacaatagcacccaaaatgcaactgcatcaataatgttcatgcaaagtttatcttctgtggttgaattttgtaatgcaagtacccacaaccaagaagcaccaaacctacagaaccagatgtgcagcctcagaagtgcatgggatgtaatcacagactctgctgactttcaccacagctttcccatgaacgggactgagcttccacctcctcccacattctcgcttgtagaggctggtgacaaagtggtctgtttagtgctggatgtgtccagcaagatggcagaggctgacagactccttcaactacaacaagccgcagaattttatttgatgcagattgttgaaattcataccttcgtgggcattgccagtttcgacagcaaaggagagatcagagcccagctacaccaaattaacagcaatgatgatcgaaagttgctggtttcatatctgcccaccactgtatcagctaaaacagacatcagcatttgttcagggcttaagaaaggatttgaggtggttgaaaaactgaatggaaaagcttatggctctgtgatgatattagtgaccagcggagatgataagcttcttggcaattgcttacccactgtgctcagcagtggttcaacaattcactccattgccctgggttcatctgcagccccaaatctggaggaattatcacgtcttacaggaggtttaaagttctttgttccagatatatcaaactccaatagcatgattgatgctttcagtagaatttcctctggaactggagacattttccagcaacatattcagcttgaaagtacaggtgaaaatgtcaaacctcaccatcaattgaaaaacacagtgactgtggataatactgtgggcaacgacactatgtttctagttacgtggcaggccagtggtcctcctgagattatattatttgatcctgatggacgaaaatactacacaaataattttatcaccaatctaacttttcggacagctagtctttggattccaggaacagctaagcctgggcactggacttacaccctgaacaatacccatcattctctgcaagccctgaaagtgacagtgacctctcgcgcctccaactcagctgtgcccccagccactgtggaagcctttgtggaaagagacagcctccattttcctcatcctgtgatgatttatgccaatgtgaaacagggattttatcccattcttaatgccactgtcactgccacagttgagccagagactggagatcctgttacgctgagactccttgatgatggagcaggtgctgatgttataaaaaatgatggaatttactcgaggtattttttctcctttgctgcaaatggtagatatagcttgaaagtgcatgtcaatcactctcccagcataagcaccccagcccactctattccagggagtcatgctatgtatgtaccaggttacacagcaaacggtaatattcagatgaatgctccaaggaaatcagtaggcagaaatgaggaggagcgaaagtggggctttagccgagtcagctcaggaggctccttttcagtgctgggagttccagctggcccccaccctgatgtgtttccaccatgcaaaattattgacctggaagctgtaaaagtagaagaggaattgaccctatcttggacagcacctggagaagactttgatcagggccaggctacaagctatgaaataagaatgagtaaaagtctacagaatatccaagatgactttaacaatgctattttagtaaatacatcaaagcgaaatcctcagcaagctggcatcagggagatatttacgttctcaccccaaatttccacgaatggacctgaacatcagccaaatggagaaacacatgaaagccacagaatttatgttgcaatacgagcaatggataggaactccttacagtctgctgtatctaacattgcccaggcgcctctgtttattccccccaattctgatcctgtacctgccagagattatcttatattgaaaggagttttaacagcaatgggtttgataggaatcatttgccttattatagttgtgacacatcatactttaagcaggaaaaagagagcagacaagaaagagaatggaacaaaattattataaataaatatccaaagtgtcttccttcttagatataagacccatggccttcgactacaaaaacatactaacaaagtcaaattaacatcaaaactgtattaaaatgcattgagtttttgtacaatacagataagatttttacatggtagatcaacaaattctttttgggggtagattagaaaacccttacactttggctatgaacaaataataaaaattattctttaaagtaatgtctttaaaggcaaagggaagggtaaagtcggaccagtgtcaaggaaagtttgttttattgaggtggaaaaatagccccaagcagagaaaaggagggtaggtctgcattataactgtctgtgtgaagcaatcatttagttactttgattaatttttcttttctccttatctgtgcagaacaggttgcttgtttacaactgaagatcatgctatatttcatatatgaagcccctaatgcaaagctctttacctcttgctattttgttatatatattacagatgaaatctcactgctaatgctcagagatcttttttcactgtaagaggtaacctttaacaatatgggtattacctttgtctcttcataccggttttatgacaaaggtctattgaatttatttgtttgtaagtttctactcccatcaaagcagctttttaagttattgccttggttattatggatgatagttatagcccttataatgccttaactaaggaagaaaagatgttattctgagtttgttttaatacatatatgaacatatagttttattcaattaaaccaaagaagaggtcagcagggagatactaacctttggaaatgattagctggctctgttttttggttaaataagagtctttaatcctttctccatcaagagttacttaccaagggcaggggaagggggatatagaggtcccaaggaaataaaaatcatctttcatctttaattttactccttcctcttatttttttaaaagattatcgaacaataaaatcatttgcctttttaattaaaaacataaaaaaa

SEQ ID NO: 2 - protein, 943 aa, Homo sapiens [sequence omitted]

SEQ ID NO: 3 - DNA, 785 bp, Homo sapiens
tctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcag

SEQ ID NO: 4 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 5 - DNA, 1633 bp, Homo sapiens
cgtggaggcagctagcgcgaggctggggagcgctgagccgcgcgtcgtgccctgcgctgcccagactagcgaacaatacagtcgggatggctaaaggtgaccccaagaaaccaaagggcaagacgtccgcttatgccttctttgtgcagacatgcagagaagaacataagaagaaaaacccagaggtccctgtcaattttgcggaattttccaagaagtgctctgagaggtggaagacggtgtccgggaaagagaaatccaaatttgatgaaatggcaaaggcagataaagtgcgctatgatcgggaaatgaaggattatggaccagctaagggaggcaagaagaagaaggatcctaatgctcccaaaaggccaccgtctggattcttcctgttctgttcagaattccgccccaagatcaaatccacaaaccccggcatctctattggagacgtggcaaaaaagctgggtgagatgtggaataatttaaatgacagtgaaaagcagccttacatcactaaggcggcaaagctgaaggagaagtatgagaaggatgttgctgactataagtcgaaaggaaagtttgatggtgcaaagggtcctgctaaagttgcccggaaaaaggtggaagaggaagatgaagaacaggaggaggaagaagaggaggaggaggaggaggaggatgaataaagaaactgtttatctgtctccttgtgaatacttagagtaggggagcgccgtaattgacacatctcttatttgagaagtgtctgttgccctcattaggtttaattacaaaatttgatcacgatcatattgtagtctctcaaagtgctctagaaattgtcagtggtttacatgaagtggccatgggtgtctggagcaccctgaaactgtatcaaagttgtacatatttccaaacatttttaaaatgaaaaggcactctcgtgttctcctcactctgtgcactttgctgttggtgtgacaaggcatttaaagatgtttctggcattttctttttatttgtaaggtggtggtaactatggttattggctagaaatcctgagttttcaactgtatatatctatagtttgtaaaaagaacaaaacaaccgagacaaacccttgatgctccttgctcggcgttgaggctgtggggaagatgccttttgggagaggctgtagctcagggcgtgcactgtgaggctggacctgttgactctgcagggggcatccatttagcttcaggttgtcttgtttctgtatatagtgacatagcattctgctgccatcttagctgtggacaaaggggggtcagctggcatgagaatatttttttttaagtgcggtagtttttaaactgtttgtttttaaacaaactatagaactcttcattgtcagcaaagcaaagagtcactgcatcaatgaaagttcaagaacctcctgtacttaaacacgattcgcaacgttctgttattttttttgtatgtttagaatgctgaaatgtttttgaagttaaataaacagtattacatttttagaactcttctctactataacagtcaatttctgactcacagcagtgaacaaacccccactccgttgtatttggagactggcctccctataaatgtg

SEQ ID NO: 6 - protein, 200 aa, Homo sapiens
MAKGDPKKPKGKMSAYAFFVQTCREEHKKKNPEVPVNFAEFSKKCSERWKTMSGKEKSKFDEMAKADKVRYDREMKDYGPAKGGKKKKDPNAPKRPPSGFFLFCSEFRPKIKSTNPGISIGDVAKKLGEMWNNLNDSEKQPYITKAAKLKEKYEKDVADYKSKGKFDGAKGPAKVARKKVEEEDEEEEEEEEEEEEEEDE

SEQ ID NO: 7 - DNA, 781 bp, Homo sapiens
gcggcggagctgtgagccggcgactcgggtccctgaggtctggattctttctccgctactgagacacggcgggtaggtccacaggcagatccaactgggagttgaagtgtgagtgagagtgaagaggaaccagcaggcttccggagggttgtgtggtcagtgactcagagtgagaaggccctcgaagtcgtcgtccctctcatgcggtgccacgcccatggaccttcttgtctcgtcacggccataactagggaggaaggagggccgaggagtggaggggctcaggcgaagctggggtgctgttgggggtatccgagtcccagaagcacctggaaccccgacagaagattctggactccccagacgggaccaggagagggacggcatgagcgacacacacaaacacagaaccacacagccagtcccaggagcccagtaatggagagccccaaaaagaagaaccagcagctgaaagtcgggatcctacacctgggcagcagacagaagaagatcaggatacagctgagatcccagtgcgcgacatggaaggtgatctgcaagagctgcatcagtcaaacaccggggataaatctggatttgggttccggcgtcaaggtgaagataatacctaaagaggaacactgtaaaatgccagaagcaggtgaagagcaaccacaagtttaaatgaagacaagctgaaacaacgcaagctggttttatattagatatttgacttaaactatctcaataaagttttgcagctttcaccaaaaaaaaaaaa

SEQ ID NO: 8 - protein, 160 aa, Homo sapiens
MRCHAHGPSCLVTAITREEGGPRSGGAQAKLGCCWGYPSPRSTWNPDRRFWTPQTGPGEGRHERHTQTQNHTASPRSPVMESPKKKNQQLKVGILHLGSRQKKIRIQLRSQCATWKVICKSCISQTPGINLDLGSGVKVKIIPKEEHCKMPEAGEEQPQV

SEQ ID NO: 9 - DNA, 20 bp, Homo sapiens
gacggcatgagcgacacaca

SEQ ID NO: 10 - DNA, 20 bp, Homo sapiens
ccatgtcgcgcactgggatc

SEQ ID NO: 11 - DNA, 28 bp, Homo sapiens
ctgaaagtcgggatcctacacctgggca

SEQ ID NO: 12 - DNA, 20 bp, Homo sapiens
ggccaccgtctggattcttc

SEQ ID NO: 13 - DNA, 20 bp, Homo sapiens
gaagaatccagacggtggcc

SEQ ID NO: 14 - DNA, 26 bp, Homo sapiens
ccgccccaagatcaaatccacaaacc

SEQ ID NO: 15 - DNA, 21 bp, Homo sapiens
atggcagaggctgacagactc

SEQ ID NO: 16 - DNA, 25 bp, Homo sapiens
ttcaaccacctcaaatcctttctta

SEQ ID NO: 17 - DNA, 27 bp, Homo sapiens
tcgacagcaaaggagagatcagagccc

SEQ ID NO: 18 - DNA, 18 bp, Homo sapiens
ttacgacccgctcagccc

SEQ ID NO: 19 - DNA, 19 bp, Homo sapiens
ctcccaacgccactgacaa

SEQ ID NO: 20 - DNA, 22 bp, Homo sapiens
ccaggccgagcccctcagaacc

SEQ ID NO: 21 - DNA, 1800 bp, Homo sapiens
gcgcctcattgccactgcagtgactaaagctgggaagacgctggtcagttcacctgccccactggttgttttttaaacaaattctgatacaggcgacatcctcactgaccgagcaaagattgacattcgtatcatcactgtgcaccattggcttctaggcactccagtggggtaggagaaggaggtctgaaaccctcgcagagggatcttgccctcattctttgggtctgaaacactggcagtcgttggaaacaggactcagggataaaccagcgcaatggattgggggacgctgcacactttcatcgggggtgtcaacaaacactccaccagcatcgggaaggtgtggatcacagtcatctttattttccgagtcatgatcctagtggtggctgcccaggaagtgtggggtgacgagcaagaggacttcgtctgcaacacactgcaaccgggatgcaaaaatgtgtgctatgaccactttttcccggtgtcccacatccggctgtgggccctccagctgatcttcgtctccaccccagcgctgctggtggccatgcatgtggcctactacaggcacgaaaccactcgcaagttcaggcgaggagagaagaggaatgatttcaaagacatagaggacattaaaaagcacaaggttcggatagaggggtcgctgtggtggacgtacaccagcagcatctttttccgaatcatctttgaagcagcctttatgtatgtgttttacttcctttacaatgggtaccacctgccctgggtgttgaaatgtgggattgacccctgccccaaccttgttgactgctttatttctaggccaacagagaagaccgtgtttaccatttttatgatttctgcgtctgtgatttgcatgctgcttaacgtggcagagttgtgctacctgctgctgaaagtgtgttttaggagatcaaagagagcacagacgcaaaaaaatcaccccaatcatgccctaaaggagagtaagcagaatgaaatgaatgagctgatttcagatagtggtcaaaatgcaatcacaggtttcccaagctaaacatttcaaggtaaaatgtagctgcgtcataaggagacttctgtcttctccagaaggcaataccaacctgaaagttccttctgtagcctgaagagtttgtaaatgactttcataataaatagacacttgagttaactttttgtaggatacttgctccattcatacacaacgtaatcaaatatgtggtccatctctgaaaacaagagactgcttgacaaaggagcattgcagtcactttgacaggttccttttaagtggactctctgacaaagtgggtactttctgaaaatttatataactgttgttgataaggaacatttatccaggaattgatacgtttattaggaaaagatatttttataggcttggatgtttttagttccgactttgaatttatataaagtatttttataatgactggtcttccttacctggaaaaacatgcgatgttagttttagaattacaccacaagtatctaaatttccaacttacaaagggtcctatcttgtaaatattgttttgcattgtctgttggcaaatttgtgaactgtcatgatacgcttaaggtgggaaagtgttcattgcacaatatatttttactgctttctgaatgtagacggaacagtgtggaagcagaaggcttttttaactcatccgtttggccgatcgttgcagaccactgggagatgtggatgtggttgcctccttttgctcgtccccgtggcttaacccttct

SEQ ID NO: 22 - protein, 261 aa, Homo sapiens
MDWGTLHTFIGGVNKHSTSIGKVWITVIFIFRVMILVVAAQEVWGDEQEDFVCNTLQPGCKNVCYDHFFPVSHIRLWALQLIFVSTPALLVAMHVAYYRHETTRKFRRGEKRNDFKDIEDIKKHKVRIEGSLWWTYTSSIFFRIIFEAAFMYVFYFLYNGYHLPWVLKCGIDPCPNLVDCFISRPTEKTVFTIFMISASVICMLLNVAELCYLLLKVCFRRSKRAQTQKNHPNHALKESKQNEMNELISDSGQNAITGFPS

SEQ ID NO: 23 - DNA, 21 bp, Homo sapiens
attccaggcgacatcctcact

SEQ ID NO: 24 - DNA, 24 bp, Homo sapiens
gtttatccctgagtcctgtttcca

SEQ ID NO: 25 - DNA, 27 bp, Homo sapiens
tgtgcaccattggcttctaggcactcc

SEQ ID NO: 26 - DNA, 2257 bp, Homo sapiens
attttgcttacagagtcccgtctcaccatcctgggcttccaacggagactgcggtatccgcggctggagacccagcggcgagtagccttttgctcccggacggacttgagaggcttaaaggatggcctcgtcagatctggaacaattatgctctcatgttaatgaaaagattggcaatattaagaaaaccttatcattaagaaactgtggccaggaacctaccttgaaaactgtattaaataaaataggagatgagatcattgtaataaatgaacttctaaataaattggaattggaaattcagtatcaagaacaaaccaacaattcactcaaggaactctgtgaatctcttgaagaagattacaaagacatagaacatcttaaagaaaacgttccttcccatttgcctcaagtaacagtaacccagagctgtgttaagggatcagatcttgatcctgaagaaccaatcaaagttgaagaacctgaacccgtaaagaagcctcccaaagagcaaagaagtattaaggaaatgccatttataacttgtgatgagttcaatggtgttccttcgtacatgaaatcccgcttaacctataatcaaattaatgatgttattaaagaaatcaacaaggcagtaattagtaaatataaaatcctacatcagccaaaaaagtctatgaattctgtgaccagaaatctctatcacagatttattgatgaagaaacgaaggataccaaaggtcgttattttatagtggaagctgacataaaggagttcacaactttgaaagctgacaagaagtttcacgtgttactgaatattttacgacactgccggaggctatcagaggtccgagggggaggacttactcgttatgttataacctgagtcccttgtgaacttttgaacataccaacagggtatagagtatagaggctatttctataattttcttatatataatttttttaacttttaatcttttttgtttccttttttttttttttgagacaggatcttgctttgtcacccaggggcttgctttgtcacgcaggctagagtgcagtggcgcaaacatggctcactgcagcctcaacctcccaggctcaagtgatcctcccacctcagccccctgaatggctgggactacaagcgtgcgccaccatgcctggctaatttttgtattttttggagagatggggtttcaccatgttgcctaggctggtcttgagctcctgagctcaaacaatccaccctcctcagcctcccaaagtgctgggattacaggcttgagccaccacacctgacctattcttgtttcttataaaaataaaacttttttggataaagcttatttcttgtttttttctttttctttttttttttttttcgagactccatctcagaaaaaaagaaaaaaagactgggtacagatgtgatattggaagaaaaagatcaagctgatgaggttaggatacccaggccctttggacttaaagatcactagtgtctaaattccatcgatggcatttcagtctataggtaaacttcctggaagctggatttggagacagtttatcatctgattattgggctttcgtataggtccttagggagcagcttacctgaaatgcatttagtgtacaccagtctgtaaacttcaacctgtaatgaaagtgtaataaatgtacattgagttgatgtgataatgtgatataataagaaatatatatttgatcttcctatctagttccttgttcagagctcctaaaacccttgtaatttccaaagtgatggagtacatcttttgttctagtatttggtctttgaccccagttcctgacacaaagctcctaaattcctttaaatttcccagtgataggagaattttttgttctaatgaggtcactcttgatgggcacctggataactcaggatgggggctgctcacaaagaccacatcatgattggaagtttcaaactttcagtctcccacctccagagaggggagaggggctggagatttgtgtcaataatccatcaggcctatgtcaacaagacataatccgttaactatggagttcagggagcttcagggttggcaaacattttgatgtgccaggaaggtgacgcactccagctttatgaagtcagcaagtcctgtgctcaggatgcttytggaccttgccccaggtaccccttcatgtggctgttgttcatctgtatcctttgtagtagccttaaaataaactgtta

SEQ ID NO: 27 - protein, 255 aa, Homo sapiens
MASSDLEQLCSHVNEKIGNIKKTLSLRNCGQEPTLKTVLNKIGDEIIVINELLNKLELEIQYQEQTNNSLKELCESLEEDYKDIEHLKENVPSHLPQVTVTQSCVKGSDLDPEEPIKVEEPEPVKKPPKEQRSIKEMPFITCDEFNGVPSYMKSRLTYNQINDVIKEINKAVISKYKILHQPKKSMNSVTRNLYHRFIDEETKDTKGRYFIVEADIKEFTTLKADKKFHVLLNILRHCRRLSEVRGGGLTRYVIT

SEQ ID NO: 28 - DNA, 22 bp, Homo sapiens
cccagagctgtgttaagggatc

SEQ ID NO: 29 - DNA, 23 bp, Homo sapiens
gttaagcgggatttcatgtacga

SEQ ID NO: 30 - DNA, 28 bp, Homo sapiens
agaacctgaacccgtaaagaagcctccc

SEQ ID NO: 31 - DNA, 1740 bp, Homo sapiens
atgaacaaactgtatatcggaaacctcagcgagaacgccgccccctcggacctagaaagtatcttcaaggacgccaagatcccggtgtcgggacccttcctggtgaagactggctacgcgttcgtggactgcccggacgagagctgggccctcaaggccatcgaggcgctttcaggtaaaatagaactgcacgggaaacccatagaagttgagcactcggtcccaaaaaggcaaaggattcggaaacttcagatacgaaatatcccgcctcatttacagtgggaggtgctggatagtttactagtccagtatggagtggtggagagctgtgagcaagtgaacactgactcggaaactgcagttgtaaatgtaacctattccagtaaggaccaagctagacaagcactagacaaactgaatggatttcagttagagaatttcaccttgaaagtagcctatatccctgatgaaacggccgcccagcaaaaccccttgcagcagccccgaggtcgccgggggcttgggcagaggggctcctcaaggcaggggtctccaggatccgtatccaagcagaaaccatgtgatttgcctctgcgcctgctggttcccacccaatttgttggagccatcataggaaaagaaggtgccaccattcggaacatcaccaaacagacccagtctaaaatcgatgtccaccgtaaagaaaatgcgggggctgctgagaagtcgattactatcctctctactcctgaaggcacctctgcggcttgtaagtctattctggagattatgcataaggaagctcaagatataaaattcacagaagagatccccttgaagattttagctcataataactttgttggacgtcttattggtaaagaaggaagaaatcttaaaaaaattgagcaagacacagacactaaaatcacgatatctccattgcaggaattgacgctgtataatccagaacgcactattacagttaaaggcaatgttgagacatgtgccaaagctgaggaggagatcatgaagaaaatcagggagtcttatgaaaatgatattgcttctatgaatcttcaagcacatttaattcctggattaaatctgaacgccttgggtctgttcccacccacttcagggatgccacctcccacctcagggcccccttcagccatgactcctccctacccgcagtttgagcaatcagaaacggagactgttcatctgtttatcccagctctatcagtcggtgccatcatcggcaagcagggccagcacatcaagcagctttctcgctttgctggagcttcaattaagattgctccagcggaagcaccagatgctaaagtgaggatggtgattatcactggaccaccagaggctcagttcaaggctcagggaagaatttatggaaaaattaaagaagaaaactttgttagtcctaaagaagaggtgaaacttgaagctcatatcagagtgccatcctttgctgctggcagagttattggaaaaggaggcaaaacggtgaatgaacttcagaatttgtcaagtgcagaagttgttgtccctcgtgaccagacacctgatgagaatgaccaagtggttgtcaaaataactggtcacttctatgcttgccaggttgcccagagaaaaattcaggaaattctgactcaggtaaagcagcaccaacaacagaaggctctgcaaagtggaccacctcagtcaagacggaagtaa

SEQ ID NO: 32 - protein, 579 aa, Homo sapiens
MNKLYIGNLSENAAPSDLESIFKDAKIPVSGPFLVKTGYAFVDCPDESWALKAIEALSGKIELHGKPIEVEHSVPKRQRIRKLQIRNIPPHLQWEVLDSLLVQYGVVESCEQVNTDSETAVVNVTYSSKDQARQALDKLNGFQLENFTLKVAYIPDETAAQQNPLQQPRGRRGLGQRGSSRQGSPGSVSKQKPCDLPLRLLVPTQFVGAIIGKEGATIRNITKQTQSKIDVHRKENAGAAEKSITILSTPEGTSAACKSILEIMHKEAQDIKFTEEIPLKILAHNNFVGRLIGKEGRNLKKIEQDTDTKITISPLQELTLYNPERTITVKGNVETCAKAEEEIMKKIRESYENDIASMNLQAHLIPGLNLNALGLFPPTSGMPPPTSGPPSAMTPPYPQFEQSETETVHLFIPALSVGAIIGKQGQHIKQLSRFAGASIKIAPAEAPDAKVRMVIITGPPEAQFKAQGRIYGKIKEENFVSPKEEVKLEAHIRVPSFAAGRVIGKGGKTVNELQNLSSAEVVVPRDQTPDENDQVVVKITGHFYACQVAQRKIQEILTQVKQHQQQKALQSGPPQSRRK

SEQ ID NO: 33 - DNA, 21 bp, Homo sapiens
catggactggctttctggttg

SEQ ID NO: 34 - DNA, 24 bp, Homo sapiens
ctgagaaaagctctggccttaaac